(19) United States
(12) Patent Application Publication
(10) Pub. No.: US 2026/0224635 A1
(43) Pub. Date: Aug. 6, 2026

(54) ENHANCED OSTEOGENIC COMPOSITION

(71) Applicant: Access2Bone IP BV, Leiden (NL)

(72) Inventors: Michael F. Sickler, Leiden (NL); Arnoldus C.M. van de Mortel, Leiden (NL); Nienke de Roode, Leiden (NL)

(73) Assignee: Access2Bone IP BV, Leiden (NL)

(21) Appl. No.: 19/032,186

(22) Filed: Jan. 20, 2025

Related U.S. Application Data
(63) Continuation-in-part of application No. 17/517,326, filed on Nov. 2, 2021, now abandoned.
Application No. 17/517,326 is a continuation of application No. 16/887,919, filed on May 29, 2020, now abandoned.
(60) Provisional application No. 62/872,364, filed on Jul. 10, 2019.

(51) Int. Cl. A61K 35/32; A61K 9/19; A61K 47/02

(52) U.S. Cl. CPC A61K 35/32; A61K 9/19; A61K 47/02

(57) ABSTRACT

An enhanced osteogenic composition is provided of connective tissue proteins having molecular weights greater than or equal to 3.5 kDa wherein the composition is prepared by treating demineralized bone material in an acidic cleavage extraction medium. When the acid is at least dihydric or dihydric or higher polyols are present, enhanced stabilization of osteoinductive proteins therein results. A method of making the enhanced osteogenic composition is also provided.

### RELATED APPLICATIONS

This application is a continuation in part application of U.S. application Ser. No. 17/517,326, filed Nov. 2, 2021, that in turn is a continuation of U.S. application Ser. No. 16/887,919, filed on May 29, 2020, that in turn claims priority benefit of U.S. Provisional Application Ser. No. 62/872,364, titled: “Enhanced Osteogenic Composition”, filed on Jul. 10, 2019, the aforementioned are hereby incorporated by reference for all purposes.

### FIELD OF INVENTION

This invention relates to a material exhibiting osteogenic activity and, more particularly, an enhanced osteogenic composition derived from demineralized bone capable of being mixed with a natural or synthetic implant and method of making same. The invention further relates to a natural or synthetic implant, e.g., allograft bone tissue and/or calcium phosphate which is mixed with or otherwise contains the enhanced osteogenic composition.

### BACKGROUND OF THE INVENTION

Demineralized bone material (“DBM”) inherently contains connective tissue proteins. These connective tissue proteins are known to be biologically active (e.g., osteoinductive) as they have shown to promote bone growth particles contained and delivered by some inert bioresorbable carrier, typically with a putty-like consistency. Bone morphogenic proteins (BMP) are exemplary of these connective tissue proteins. BMP-2 and BMP-7 are part of the TGF-beta superfamily of proteins and are used to clinically treat fractures and promote bone graft integration. However, recombinant versions of these proteins have unacceptably short functional half-lives in vivo, as compared to those provided from DBM. While there have been numerous attempts to secure high yields of these proteins using concentrated mineral acid extractions, as for example detailed in U.S. Pat. No. 5,236,456, these approaches met with limited success owing low yields of active proteins. As the field as historically assumed that osteoinductive potential of a bone repair substance correlates with the total amount of osteoinductive constituent(s) that are present, DBM extraction has remained an activate area of research as the dense DBM matrix is widely seen as interfering with osteoinductive proteins therein being able to bind to subject cell receptors so as to stimulate bone growth in a subject

Subsequent efforts have attempted to use extract citric acid or malic acid, or acetic acid to extract BMPs from DBM, yet with limited improvements in BMP yields while producing either biphasic materials or crumbly solids.

Thus, there remains a need for an enhanced osteogenic composition with greater concentrations of osteoinductive constituents relative to the prior art. There further exists a need for the resulting composition to have a malleable, putty-like form that is amenable to clinical application to an in vivo orthopedic situs in need of osteoinduction.

### BRIEF DESCRIPTION OF THE DRAWINGS

FIG. 1 is a photograph (100 times magnification) of a section of animal living tissue treated with the Enhanced Osteogenic Composition of the present invention and demineralized bone material as described below in Example 14; and

FIG. 2 is a photograph (100 times magnification) of a section of animal living tissue treated with demineralized bone material only as described below in Example 14.

### DESCRIPTION OF THE PREFERRED EMBODIMENT

The use of osteogenic composition derived from demineralized cortical cancellous and/or corticocancellous autogenic, allogeneic or xenogeneic bone (hereinafter referred to as “Bone”) in surgical bone repairs and/or reconstruction has been known for decades. The conventional production of such osteogenic composition begins with pulverization of the Bone resulting in Bone particles generally ranging from 100 microns to 300 microns in average particle size. Thereafter, these pulverized Bone particles are defatted and disinfected using a suitable solvent (e.g., ethanol or the like). Once the pulverized Bone particles have been defatted and disinfected, they are immersed in acid over time to effect demineralization. The demineralization process removes essentially all of the original calcium content of the Bone resulting in a demineralized bone material containing at least less than about 10% weight calcium with the preferred range from about 0.5% to 1% weight percent calcium. The term “about” used herein this specification is defined as plus or minus 1%. The demineralized Bone particles are then subject to an acid-promoted cleavage process using acid in the concentration of 2M to 3M for a few minutes to several hours either in ambient temperature (e.g., below about 25° C.) or a heated environment to a temperature of about 30° C. to about 75° C., resulting in a phase change from solid to a viscous liquid of osteogenic composition. This liquid osteogenic composition can then be further processed via titration, dialysis, and lyophilization into dry powder for easy handling and storage. See e.g., U.S. Pat. No. 5,236,456; Section E Processing and Storage, Standards for Tissue Banking, 14th Edition (2016), American Association of Tissue Banks.

The present invention provides an osteogenic composition exhibiting enhanced and/or improved osteogenic activity (hereinafter referred to as “enhanced osteogenic composition” or “EOC”) when compared to currently commercially available osteogenic compositions (e.g., the ones described in U.S. Pat. No. 5,236,456). EOC's enhanced and/or improved osteogenic activity is highly desired for use in surgical bone repairs and/or reconstruction. The present invention further provides a method of making the EOC as described below.

It is to be understood that in instances where a range of values are provided that the range is intended to encompass not only the end point values of the range but also intermediate values of the range as explicitly being included within the range and varying by the last significant figure in the range. By way of example, a recited range from 1 to 4 is intended to include 1-2, 1-3, 2-4, 3-4, and 1-4.

### I. Method of Making Enhanced Osteogenic Composition

In one exemplary embodiment of the present invention, EOC is prepared by the following method: (1) pulverizing Bone using art-disclosed means as discussed above (preferably the Bone particles are ranging from 110 microns to 850 microns); (2) defatting and disinfecting the pulverized Bone particles using art-disclosed means as discussed above; and (3) demineralizing the pulverized Bone particles by immersing them in acid over time resulting in a demineralized bone material. It is preferred that the demineralized bone material shall contain no greater than about 10, no greater than about 1 and most preferably no greater than about 0.5 weight percent calcium.

Acids which can be used in the demineralization step include inorganic acids such as hydrochloric acid and organic acids such as peracetic acid. The concentration of the acid in this demineralization step is preferred to be about 0.5M to about 1.0M but other art-disclosed concentration(s) may be used. The amount of time for such acid immersion is preferred to be from about 20 minutes to about 2 hours under ambient conditions. However, acid immersion time is not limited to this range and may be shorter or longer as long as essentially all of the original calcium content of the Bone is removed during this demineralization step, resulting in a demineralized bone material (“DBM”) containing at least less than about 10% weight calcium with the preferred range from about 0.5% to 1% weight percent calcium.

After the demineralization step, the method of the present invention further includes subjecting the DBM to an acid-promoted cleavage step (i.e., treating the DBM in an acidic extraction medium) performed at a temperature controlled environment (i.e., extraction temperature) of greater than 25° C. but less than 30° C., preferably from 26° C. to 29° C. The temperature ranges of between 26° C. to 37° C., between 26° C. to 35° C., between 26° C. to 50° C., and between 26° C. to 80° C. are also included in the present invention.

While the acid-promoted cleavage step can utilize the same acid used in the demineralization step, it is preferred that an organic acid such as Ascorbic, Malic, Citric, Tartaric and/or combination thereof is used for the acid-promoted cleavage step in a concentration of at least about 3.0M to about 5.0M, preferably about 3.3M to about 4.4M, most preferable about 3.5M to about 4.2M. The preferred pH during this acid-promoted cleavage step is between 0.10 to 0.45, more preferably between 0.3 to 0.45, and most preferable between 0.35 to 0.45. Depending on the desired molarity, the acid treatment can have a ratio of acid to DBM at least about 1 mL/g (of DBM) to about 100 mL/g, preferably about 5 mL/g to 50 mL/g, most preferable about 10 mL/g to about 20 mL/g. The acid treatment time can vary depending on average particle size(s) of the DBM and the temperature. Preferably the acid treatment time is from about 20 minutes to about 144 hours, more preferably about 24 hours to about 120 hours, and most preferably about 48 hours to 72 hours, being sufficient to yield the desired results. The acid treatment time from about 20 minutes to about 240 hours, from about 24 hours to about 144 hours, and from about 48 hours to about 120 hours are also included in the present invention.

Other organic acids operative herein illustratively include quinic acid, shikimic acid, and combinations thereof.

It has been surprisingly found that stabilization of the osteoinductive forms of BMPs, as opposed to inactive or denatured forms thereof, is dependent on the type of acid used in the acid-promoted cleavage step. According to the present invention, osteoinductive forms of BMPs isolated from DMB are stabilized by the presence of dihydric, trihydric, and polyhydric acid salts relative to monohydric acid salts. As a result, tartaric acid (dihydric) and ascorbic acid (tetrahydric) are more effective than citric and malic acids (both monohydric). In still other inventive embodiments, dihydric, trihydric, polyhydric alcohols; neutralized acid salts that are dihydric, trihydric, polyhydric; or combinations thereof are added to the composition during, or after the acid-promoted cleavage step. Without intending to be bound to a particular theory, the hydroxyl groups thereof substitute for water at the osteoinductive protein surface, conferring thermodynamic stability by limiting thermal unfolding. As a result, in some inventive embodiments, dihydric, trihydric, polyhydric alcohols; neutralized acid salts that are dihydric, trihydric, polyhydric; or combinations thereof are present in an amount of at least 0.1 percent by weight of the composition produced by the acid-promoted cleavage step. In still other embodiments, the acid-promoted neutralized acid salts are present from 0.1 to 5.0 total weight percent of the composition. These polyhydric molecules appear to interact with hydrophilic surface regions of the osteoinductive proteins in an inventive composition.

The present invention retains high levels of osteoinductive protein activity compared to conventional techniques. In some inventive embodiments, BMP-2 is present in an inventive composition in an amount of greater than 50 nanograms per gram (ng/g) of DBM exposed to acid-promoted cleavage. In still other embodiments, BMP-2 is present from 60 to 145 ng/g. In still other embodiments, BMP-7 is present in an inventive composition in an amount of greater than 300 ng/g of DBM exposed to acid-promoted cleavage. In still other embodiments, BMP-2 is present from 500 to 900 ng/g. In still other embodiments, both BMP-2 and BMP-7 are present in an inventive composition. In still other inventive embodiments, BMP-2 and BMP-7 are present in amounts of at least 50 ng/g and 300 ng/g, respectively.

It should be noted that as an alternative embodiment of the present invention, the demineralization step and the acid-promoted cleavage step can be carried out simultaneously (i.e., not separately) using the same acid(s), temperature(s), and pH(s).

The acid-promoted cleavage step yields a viscous liquid composition, which still retains a high concentration of organic acid so it can be titrated/diluted using a suitable basic substance and/or liquid and known techniques until a pH of about 3 is obtained. At this pH, the hydrolytic degradation of the proteins caused by the acid is stopped.

Alternatively, and/or additionally, the viscous liquid composition can be subjected to a dialysis step using a Regenerated Cellulose Membrane filter between 1.0 kilodaltons (kDa) to 12.0 kDa, preferably greater or equal to 3.5 kDa, more preferably between 3.5 kDa to 6.0 kDa, and most preferably 3.5 kDa to 4 kDa. In addition to using deionized water alone, a buffer solution of calcium carbonate and/or calcium hydroxide can be used for dialysis to drive pH to a neutral solution. Dialysis using a Regenerated Cellulose Membrane filter provides a molecular separation for desalting and isolating proteins. After the diluting step and/or the dialysis step, EOC of the present invention is provided. During storage, at room or above freezing temperatures, the EOC remains in viscous liquid form having a viscosity such as about 10 to 16,000 centipoise. The molecular weight of the EOC is equal or greater than 3.5 kDa. The EOC will remain in viscous liquid form if it is maintained in a sealed container at room temperature.

Alternatively, the EOC can be lyophilized using known techniques to yield a dry product. Thereafter, the lyophilized product (i.e., dry EOC) can be hydrated, as needed, into a suitable form for mixing or injection into desired treatment sites such as tissue, implants, or the like.

The EOC, whether dry, liquid or in solution for injection, serves as an excellent host of medically/surgically useful substances including insoluble solids such as demineralized bone powder, collagen and insoluble collagen derivatives, hydroxyapaptite, etc., and soluble solids and/or liquids dissolved therein. The above EOC can be used in many ways to promote bone growth. For example, EOC can be provided in the form a dry, flowable and/or mixable substance within a natural and/or synthetic implant. Regardless of the physical form of the EOC, stabilization is enhanced by retaining neutralized acid salts that are dihydric, trihydric, polyhydric; alone or in combination with dihydric, trihydric, polyhydric alcohols.

### Example 1 24 Hour Acid-Promoted Cleavage Using Tartaric Acid pH 0.1-0.2

In one exemplary embodiment of the present invention, the method of making EOC described above is used with the following changes. The DBM is sieved to an average particle size of from about 110 microns to about 850 microns. The DBM is then introduced into a 250 mL beaker for the acid-promoted cleavage process wherein a solution of Tartaric acid with a pH of 0.15 (4.2 M) is then added to the beaker using 11 mL/g wherein g is the measurement unit for the DBM. During the acid-promoted cleavage process, the Tartaric acid solution containing the DBM is subjected to a controlled temperature of 29° C. for 24 hours using an orbital mixing motion between 10 to 500 RPM (e.g., 220 RPM), preferably between 100 to 400 RPM, and more preferably between 150-250 RPM.

### Example 2 48 Hour Acid-Promoted Cleavage Using Tartaric Acid pH 0.1-0.2

In another exemplary embodiment of the present invention, the method of making EOC described above is used with the following changes. The DBM is sieved to an average particle size of from about 110 to about 850 microns. This DBM is then introduced into a 250 ml beaker for the acid-promoted cleavage process wherein a solution of Tartaric acid with a pH of 0.15 (4.2 M) is then added to the beaker using 11 mL/g wherein g is the measurement unit for the DBM. During the acid-promoted cleavage process, the Tartaric acid solution containing the DBM is subjected to a controlled temperature of 29° C. for 48 hours using an orbital mixing motion between 10 to 500 RPM (e.g., 220 RPM), preferably between 100 to 400 RPM, and more preferably between 150-250 RPM.

### Example 3 72 Hour Acid-Promoted Cleavage Using Tartaric Acid pH 0.3-0.4

In yet another exemplary embodiment of the present invention, the method of making EOC described above is used but with the following specifications. The DBM is sieved to an average particle size of from about 110 to about 850 microns. This DBM is then introduced into a 250 ml beaker for the acid-promoted cleavage process wherein a solution of Tartaric acid with a pH of 0.35 (3.5 M) is then added to the beaker using 20 mL/g wherein g is the measurement unit for the DBM. During the acid-promoted cleavage process, the Tartaric acid solution containing the DBM is subjected to a controlled temperature of 29° C. for 72 hours using an orbital mixing motion between 10 to 500 RPM (e.g., 220 RPM), preferably between 100 to 400 RPM, and more preferably between 150-250 RPM.

### Example 4 96 Hour Acid-Promoted Cleavage Using Tartaric Acid pH 0.35-0.45

In one exemplary embodiment of the present invention, the method of making EOC described above is used but with the following specifications. The DBM is sieved to an average particle size of from about 110 to about 850 microns. This DBM is then introduced into a 250 mL beaker for the acid-promoted cleavage process wherein a solution of Tartaric acid with a pH of 0.40 (3.3 M) is then added to the beaker using 20 mL/g wherein g is the measurement unit for the DBM. During the acid-promoted cleavage process, the Tartaric acid solution containing the DBM is subjected to a controlled temperature of 29° C. for 96 hours using an orbital mixing motion between 10 to 500 RPM (e.g., 220 RPM), preferably between 100 to 400 RPM, and more preferably between 150-250 RPM.

### Example 5 Neutralization of Osteogenic Composition

The viscous liquid composition described above in Example 3 and Example 4 is diluted to 25 mL/g using deionized water or the like and dialyzed using a Regenerated Cellulose Membrane of 3.5 kDa (pore size) against deionized water until the pH of the EOC reaches a pH range between 4.50 to 7.0, preferably between 4.75 to 7.0, and more preferably between 5.0 to 7.0. Please note that “g” (i.e., gram) is the measurement unit for the DBM originally introduced to the acid-promoted cleavage process resulting in the viscous liquid composition.

### Example 6 Neutralization of Osteogenic Composition

The viscous liquid composition described above in Example 3 and Example 4 is diluted to 25 mL/g using deionized water or the like and dialyzed using a Regenerated Cellulose Membrane of 6 kDa (pore size) against deionized water until the pH range between 4.50 to 7.0, preferably between 4.75 to 7.0, and more preferably between 5.0 to 7.0.

### Example 7 Neutralization of Osteogenic Composition

The viscous liquid composition described above in Example 1, 2, 3 and 4 is directly dialyzed (i.e., undiluted) using a Regenerated Cellulose Membrane of 3.5 kDa (pore size) against deionized water until the pH range between 4.50 to 7.0, preferably between 4.75 to 7.0, and more preferably between 5.0 to 7.0.

### Example 8 Neutralization of Osteogenic Composition

The viscous liquid composition described above in Example 1, 2, 3 and 4 is directly dialyzed (i.e., undiluted) using a Regenerated Cellulose Membrane of 6 kDa (pore size) against deionized water until the pH range between 4.50 to 7.0, preferably between 4.75 to 7.0, and more preferably between 5.0 to 7.0.

### Example 9 Dry Osteoinductive and Osteoconductive Granule

The EOC described above in Example 5, 6, 7 and 8 still in viscous liquid form would be mixed with a synthetic calcium phosphate powder, by undergoing a solid state reaction, and this mixture will turn into a solid, which will then be grinded down to a granule. This granule could be in the form of an Apatite with a porosity of 30% to 90%, with a granules size of 500 to 700 micron.

### Example 10 Osteoinductive and Osteoconductive Flowable Bone Repair Composition

The EOC described above in Example 5, 6, 7 and 8 still in a viscous liquid form would be mixed through a carrier of nano-sized calcium phosphate particles suspended in an aqueous solution.

### Example 11 Dry EOC Particles

The EOC described above in Example 5, 6, 7 and 8 still in a viscous liquid form is casted in a PETG tray with or without wells or other suitable casting materials ranging from 1-2500 mL in volume. Once casted, the casting trays or similar are placed in a deep freeze at about −40° C. to −80° C. for a minimum of 24 hours, then lyophilized over multiple days to remove excess moisture to form a lyophilized dried EOC. This lyophilized EOC can be blended into small particles to create a “pixie dust” like product having an average particle size of between 0.1 microns to 2000 micron (“EOC Particles” and use for surgical implantation or mixed with additional active or inactive fillers.

Additionally, cancellous bone and/or demineralized bone matrix/powder may also be added, either alone as particles or in combination with one another, to the dried EOC Particles. After terminal sterilization, this material may be used for insertion at, in, on, or near a bone and/or chondral defects.

In addition, the lyophilized dried EOC Particles or prior to blending into particulate form can be hydrated using deionized water or buffer saline at a concentration up to 10.0 mL/g. This viscous mixture is then poured in a tray with the desired shape and volume. The above freezing and lyophilization steps are repeated. This composition, before or after hydration, may be used for injection or surgical implantation at bone defect sites or mixed with additional active or inactive fillers i.e., collagen sponge, demineralized bone matrix/powder, CaP, etc.

### Example 12 Dry EOC Matrix Booster

The EOC described above in Example 5, 6, 7 and 8 still in a viscous liquid form, prior to the freezing step described above, can be mixed with cancellous chips, demineralized bone fibers, cancellous sponge, demineralized bone matrix chips and demineralized bone matrix granules. The combined product is then frozen and lyophilized. These materials may be used for surgical implantation at, in, on, or near the bone and/or chondral defect sites.

### Example 13 Dry Fibrous EOC Matrix with Instant Imbibition

The EOC described above in Example 5, 6, 7 and 8 still in a viscous liquid form is casted in a Polypropylene (PP) Cup up to 50 mL then placed inside an aluminum alloy lyophilization tray with Tyvek cover (lid). The lyophilization tray is then placed in a deep freeze at about −40° C. to −80° C. for a minimum of 24 hours, and then lyophilized over multiple days to remove excess moisture to form a lyophilized dry fibrous matrix (“Fibrous EOC Matrix”). After terminal sterilization, using gamma or ebeam (or similar methods), the lyophilized Fibrous EOC Matrix can be hydrated using a buffered saline, saline, blood, bone marrow then mixed with additional active or inactive fillers i.e., collagen sponge, demineralized bone matrix/powder, CaP, etc. These materials may be used for surgical implantation at, in, on, or near the bone and/or chondral defect sites. The Fibrous EOC Matrix has instant imbibition (absorption) properties defined as having the ability to absorb in less than 60 seconds (i.e., having a range of from 1 second to 60 seconds, preferably from 5 seconds to 30 seconds, and more preferably from 10 seconds to 20 seconds).

### Example 14 Histopathology

In this study, the viscous liquid composition described above in Example 2 is diluted to 25 mL/g using deionized water or the like and dialyzed using a Regenerated Cellulose Membrane of 3.5 kDa (pore size) against deionized water until the pH of the EOC reaches a pH range between 4.50 to 7.0, preferably between 4.75 to 7.0, and more preferably between 5.0 to 7.0. Please note that “g” (i.e., gram) is the measurement unit for the DBM originally introduced to the acid-promoted cleavage process resulting in the viscous liquid composition.

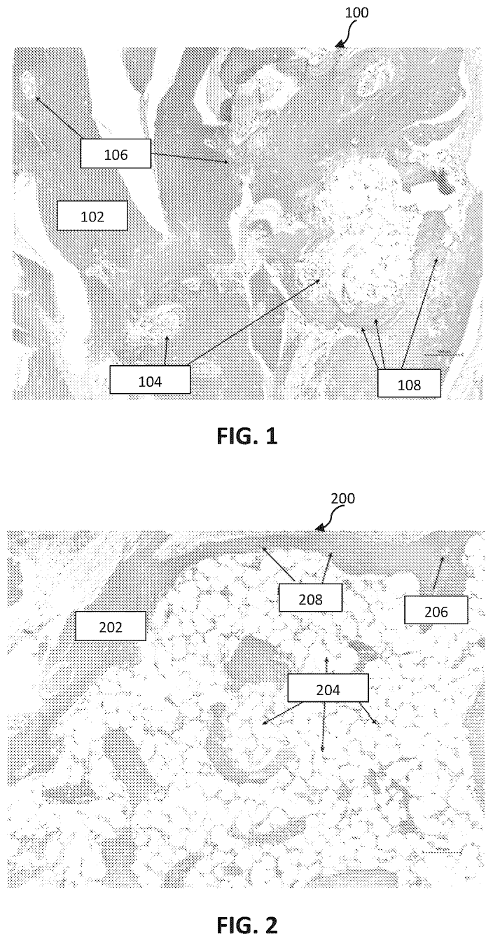

DBM is also used in this study. Approximately 0.1 gram of the EOC described above in this example is combined with approximately 0.1 gram of DBM (“EOC Sample”) is then implanted into each of the biceps femoris muscles of eight (8) athymic male rats (“animal”). The EOC includes approximately 70 ng/g of BMP-2 and 630 ng/g of BMP-7. For control purposes, approximately 0.2 gram of DBM (from the same lot/source as the DBM used in the EOC Samples) was also implanted into the left biceps femoris muscles of four (4) animals. During implantation, each animal was anesthetized and prepared for surgery. A pocket was created in each biceps femoris muscle using sharp and blunt dissection. After the incision was made, either the control DBM sample or the EOC Sample was placed into the muscle pocket, and then the muscle pocket and skin were sutured closed. After the surgery, the animals were observed daily for their general health status for twenty-eight (28) days. During this period, the animals gained weight and no abnormal clinical signs were noted for any of the animals. After twenty-eight (28) days, the animals were sacrificed when all implants sites were positively identified and harvested as study samples. Evaluation of these study samples showed that the DBM only study samples (sites implanted with 0.2 gram of DBM only) scored on a “1” for osteoinductivity, which is defined as 0 to 25% new bone formation as shown in FIG. 1. Referring to FIG. 1, which is is a representative histopathology photograph of one DBM only study sample 100 taken at 100×total magnification showing the EOC 102, the bone marrow 104, the cartilage 106, and the new bone formation 108 of the sample 100 implant site.

The EOC study samples (sites implanted with 0.1 gram of EOC and 0.1 gram of DBM) scored a “4” for osteoinductivity, which is defined as 75% to 100% new bone formation as shown in FIG. 2. Referring to FIG. 2 which is a representative histopathology photograph of one EOC study sample 200 (out of the sixteen samples) taken at 100×total magnification showing the EOC 202, the bone marrow 204, the cartilage 206, and the new bone formation 208 of the sample 200 implant site. The study's results demonstrated osteoinduction potential of the EOC in the intramuscular implant sites using the male athymic nude rat model. It should be noted that while the absolute scores of the study samples may vary from animal to animal, the study clearly indicates the EOC study samples met the histological criteria for evidence of osteoinduction as elements of new bone formation were observed in all sixteen samples and had an increase of new bone formation when compared to the control DBM.

### Example 15 Comparison of Acids as EOC Properties

An experiment was conducted to observe differences in acid-base neutralization reactions between four organic acids on Calcium carbonate (CaCO₃) as a model for bone. The four organic acids used in this study were: tartaric acid, ascorbic acid, citric acid, and malic acid. The study was conducted using calibrated lab grade instruments and reagent grade acid chemicals. The study captured reaction differences between the four organic acids. All four acids were mixed with deionized/reverse osmosis water to create acidic solutions that were equal in molarity. After completing the acid solubilization step, an equivalent weight of powdered calcium carbonate was introduced to all four organic acids at the same time and mixed thoroughly to ensure homogenous mixtures. A timer was initiated, and the study was commenced, noting visual appearance and consistency over time.

Observations were as follows:
All organic acids were fully solubilized except ascorbic acid which retained a slight white cloudy appearance and slightly incomplete solubilization.
After approximately 9 minutes all 4 acids reacted differently to the addition of the base from a visual perspective.
After approximately 12 minutes, all four organic acid-base reactions had a distinctly different texture. The textures and appearances were dissimilar amongst all four organic acids reacted with the calcium carbonate. The range of different appearance was from a slightly colored liquid covered by a light, thin foam to a “whipped crème” appearance with much more structure (resistance to flow when the container was tilted).

After approximately 13 minutes, all four organic acid-base reaction products were distinctly different from each other regarding visual and structural/textural composition.

Based on the observations it was concluded that of the four organic acids studied, the acid-base neutralization reactions with calcium carbonate reacted differently in consistency and appearance. With all other elements (amounts of water and calcium carbonate) being equal, the differences in the resultant reaction products were distinct and due entirely to the specific acid reaction.

The ability of the tartaric acid and ascorbic acid to form homogenous foams when contacting calcium carbonate as a model for a bone matrix from which EOC is extracted confers advantages as to the handling of the EOC liberated from bone. These results are summarized in Table 1.

TABLE 1
Observations of neutralized calcium carbonate as a function
of time for tested acids.

| Time(Min) | 9:19 | 11:55 | 13:17 |
|---|---|---|---|
| Citric acid | Foam with | Foam, More Clear | Yellow Liquid with some |
| | Yellow Liquid | Yellow Liquid | Top Layer Foam |
| | Below | Forming Below | |
| Malic acid | Runny Paste | Pasty, White | Chalky, Crumbly, Semi- |
| | | | Hard, White |
| Tartaric | Thick Foam | Whipped Creme, | Gel-Like, White |
| acid | | White | |
| Ascorbic | Very Foamy | Very Foamy | Very Foamy, White, White |
| acid | | | Liquid Forming Below |

### Example 16 48 Hour Acid-Promoted Cleavage Using Ascorbic Acid

The procedure of EXAMPLE 2 is repeated with a like molar amount Ascorbic acid in place of Tartaric acid. The resulting EOC is noted to form a foamed substance containing approximately 75 ng/g of BMP-2 and 755 ng/g of BMP-7 based on the amount of treated DBM. The EOC contained over 1000 ng/g of ascorbate.

### Example 17 Polyhdric Stabilization of EOC

The EOC of EXAMPLE 16 is dialyzed against 0.01 M ethanol in a conventional protein stabilization buffer to displace ascorbate associated with the osteoinductive proteins. The dialyzed composition is noted to have reduced levels of active BMP-2 and BMP-7 after two weeks of storage at 4° C. In contrast, levels of BMP-2 and BMP-7 are maintained when 1000 ng/g of glycerol is added after the dialysis and two weeks storage at 4° C.

### Example 18 Ankle-Foot Fusion Results

The composition of Example 10 is hydrated with either of sterile water, sterile saline, or the patient's bone marrow aspirate (BMA), DBM particles and bone fibers are then added and mixed thoroughly; that process taking less than 3 minutes at the instrument table in the O.R. 25 patients, 9 male and 16 female were studied, at the time of surgical intervention, the average was 57.6 years of age (range 25-84 y.o.) and a total of 48 joints were fused. The fusion procedures performed included triple arthrodesis, subtalar arthrodesis, medial column stabilization, hallux valgus corrections, and metatarsophalangeal arthrodesis, (primary and revision) bone grafts.

The resultant graft produced by slurrying the composition of Example 10 is a contiguous, malleable putty-like mass that easily delivers to the prepared fusion site. When bone marrow aspirate is used, the consistency is the same as when saline or water is used, with a robust, uniform composite graft resulting that is easily delivered to the prepared fusion site without disassociation or liquifying,

The volume of the slurried the composition of Example 10 used for all patients was ~4.5 cc.; two patients used ~3 cc. as they were small joint fusion patients and did not have fibers as part of the bone graft composite. The patient's age, assessment of the quality of the autograft harvest, the second site surgery, and the prognosis of healing to successful fusion are considerations for autograft harvesting when an autograft is needed to fill large voids associated with the fusion procedure.

The patients in our series achieved a 91.6% assessment of clinical healing with no multi-joint non-unions, no infections, nor complications warranting re-operation; no heterotopic ossification nor overdosing of the graft has been observed. Solid fusions or definite progression to fusion are observed at six months post-operative and all but 2 patients have, or are, expected to reestablish normal function based on the complete or progression to complete resolution. Only one patient has signs of a hypertrophic non-union at 5.5 months post-operative but may be progressing to completion. In contrast to the use of the composition of Example 10, a 10-20% non-union rate is commonly reported for primary arthrodesis with higher rates reported for revision patients performed without the use of the inventive composition.

Results post-operative evaluations indicate that the inventive composition-based composite graft performs to aid in bone regeneration and repair in common instrumented foot and ankle surgical procedures and serves as a potent osteoinductive bone graft substance.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An enhanced osteogenic composition comprising:
osteoinductive connective tissue proteins having molecular weights greater than or equal to 3.5 kDa and comprising at least one of BMP-2 and BMP-7;
the demineralized bone treated by an acidic extraction medium; and
at least one of a dihydric, a trihydric, a polyhydric alcohol, a neutralized acid salt that is dihydric, trihydric, polyhydric, or a combination thereof.

2. The enhanced osteogenic composition of claim 1 wherein the acidic extraction medium is a dihydric, a trihydric, or a polyhydric acid.

3. The enhanced osteogenic composition of claim 1 wherein the acidic extraction medium is a dihydric acid.

4. The enhanced osteogenic composition of claim 3 wherein the dihydric acid is tartaric acid.

5. The enhanced osteogenic composition of claim 1 wherein the acidic extraction medium is a polyhydric acid.

6. The enhanced osteogenic composition of claim 3 wherein the polyhydric acid is ascorbic acid.

7. The enhanced osteogenic composition of claim 1 wherein the acidic extraction medium has a ratio of acid to the demineralized bone material of at least about 1 m L/g to about 100 mL/g.

8. The enhanced osteogenic composition of claim 1 wherein at least one of the dihydric, the trihydric, or the polyhydric alcohol is present.

9. The enhanced osteogenic composition of claim 1 wherein the dihydric alcohol, the trihydric alcohol, the polyhydric alcohols, or the neutralized acid are present in an amount of at least 0.1 percent by weight of the composition.

10. The enhanced osteogenic composition of claim 9 wherein the amount is from 0.1 to 5.0 total weight percent of the composition.

11. The enhanced osteogenic composition of claim 1 wherein the demineralized bone material has an average particle size from about 110 microns to about 850 microns.

12. The enhanced osteogenic composition of claim 1 wherein the composition is further prepared by titration and the composition has a pH greater than or equal to 4.5.

13. The enhanced osteogenic composition of claim 1 wherein both the BMP-2 and the BMP-7 are present.

14. The enhanced osteogenic composition of claim 1 wherein both the BMP-2 and the BMP-7 are present in amounts of more than 50 nanograms per gram of demineralized bone (ng/g) and more than 300 ng/g, respectively.

15. The enhanced osteogenic composition of claim 1 is in the form of a foam or a gel.

16. A method of preparing an enhanced osteogenic composition, the method comprising:
treating demineralized bone material in an organic acidic extraction medium of at least one one of a dihydric, a trihydric, or a polyhydric organic acid, alone or in combination with one of a dihydric, a trihydric, a polyhydric alcohol to yield the enhanced osteogenic composition; and
modifying the enhanced osteogenic composition to a pH of between 3.0 and 7, the enhanced osteogenic composition containing connective tissue proteins with molecular weights greater than or equal to 3.5 kDa and containing a neutralized acid salt that is dihydric, trihydric, polyhydric, or a combination thereof;
wherein the connective tissue proteins comprise at least one of BMP-2 and BMP-7.

17. The method of claim 16 wherein the acidic extraction medium is tartaric acid.

18. The method of claim 16 wherein the predetermined time period is between 48 hours to 72 hours.

19. The method of claim 16 further comprising dialyzing the enhanced osteogenic composition with a Regenerated Cellulose Membrane.

20. The method of claim 16 further comprising mixing the enhanced osteogenic composition with calcium phosphate powder.
